(19) United States
(12) Patent Application Publication
(10) Pub. No.: US 2008/0305148 A1
(43) Pub. Date: Dec. 11, 2008

(54) Treatment of spinal injuries using human umbilical mesenchymal stem cells

(75) Inventor: Yu-Show Fu, Taipei (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: NATIONAL YANG MING UNIVERSITY, Taipei (TW)

(21) Appl. No.: 12/047,417

(22) Filed: Mar. 13, 2008

Related U.S. Application Data
(60) Provisional application No. 60/895,510, filed on Mar. 19, 2007.

(51) Int. Cl. A61F 2/00; A61K 35/12; A61P 25/00

(52) U.S. Cl. 424/426; 424/93.7

(57) ABSTRACT

Transplantation of human umbilical mesenchymal stem cells (HUMSCs) to an area of a spinal injury is therapeutically effective in treating the spinal injury. Methods for treating spinal injuries based on such transplantation are described.

### CROSS-REFERENCE TO RELATED APPLICATION

This application claims the benefit under 35 U.S.C. § (e) of U.S. Provisional Patent Application No. 60/895,510, filed Mar. 19, 2007, the disclosure of which is hereby incorporated by reference herein in its entirety.

### BACKGROUND OF THE INVENTION

Human umbilical mesenchymal stem cells (HUMSCs) are the stem cells from the mesenchymal tissue of the umbilical cord, such as Wharton's jelly (Fu YS et al., Stem Cells 2004; 24: 115-124). They can be easily obtained and processed as compared to embryonic and bone marrow stem cells. It was reported that HUMSCs could be induced to differentiate into neuron-like cells (about 87%), expressing neurofilament, functional mRNAs responsible for the syntheses of subunits of the kainate receptor and glutamate decarboxylase, and generating an inward current in response to evocation by glutamate (Fu et al., Journal of Biomedical Science 2004; 11: 652-660). It was also reported that HUMSCs were capable of differentiating into osteogenic, chondrogenic, adipogenic, and myogenic cells in vitro (Wang et al., Stem Cells 2004; 22: 1330-1337).

Currently, the combinatory treatment strategy has been used to address multiple impediments to regeneration of injured axons in the adult mammalian spinal cord. For example, the combination of Schwann cells bridge (SCs bridge), olfactory ensheathing glia and chondroitinase ABC derived from Proteus vulgaris has been used to provide significant benefit compared with graft only or the untreated group. The significant benefit has been observed both in the Basso, Beattie, and Bresnahan score (BBB score) and in the forelimb/hindlimb coupling. The significant benefit is accompanied by increased numbers of both myelinated axons in the SCs bridge and serotonergic fibers that grow through the bridge and into the caudal spinal cord (Fouad et al., The Journal of Neuroscience, 2005, 25 (5):1169-1178).

There remains a need for additional methods of treating spinal injuries. The present invention relates to such a new method.

### BRIEF SUMMARY OF THE INVENTION

It is now discovered that delivering of HUMSCs to an area of a spinal injury is therapeutically effective in treating the spinal injury.

In one general aspect, the present invention relates to a method of treating a spinal injury in a subject comprising delivering an effective amount of human umbilical mesenchymal stem cells (HUMSCs) to an area of the spinal injury.

In a preferred embodiment, the HUMSCs are obtained from Wharton's Jelly of the subject.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the embodiments shown in the drawings.

In the drawings:

FIG. 1 is a scheme showing the experimental design of an embodiment of the present invention, where HUMSCs are delivered to a spinal cord after the removal of the thoracic 8 segment of the spinal cord.

FIG. 2 comprises FIGS. 2A-2D and are enlarged images of the cross-sections of the dorsal column with (stem cell group) and without (control group) the transplantation of HUMSCs obtained from Wharton's Jelly: FIGS. 2A (control group) and 2B (stem cell group) show the rostral stumps of the spinal cord; and FIGS. 2C (control group) and 2D (stem cell group) show the caudal stumps.

FIG. 3 comprises FIGS. 3A-3F and are images showing the regeneration of nervous fibers in the spinal cord with (stem cell group) and without (control group) and the transplantation of HUMSCs obtained from Wharton's Jelly: FIGS. 3A-3C show that the neurofilament-positive fiber was rarely found in the lesion site of transected spinal cord in the control group; and FIGS. 3D-3F show that the neurofilament-positive fibers were found to grow in the lesion site of transected spinal cord in the stem cell group.

FIG. 4 is a bar graph illustrating the growth of neurofilament-positive fibers in the lesion site of the transected spinal cord with (stem cell) and without (control) the transplantation of HUMSCs, wherein * represents p<0.05.

FIG. 5 comprises FIGS. 5A-5D and are images showing the distribution of HUMSCs in rats 4 months after the transplantation of HUMSCs: FIG. 5A shows a lower magnification photomicrograph; FIGS. 5B-D show the higher magnification photomicrographs, wherein the arrows indicate anti-human specific nuclear-Ag positively stained cell-bodies.

FIG. 6 comprises FIGS. 6A-6E and are line drawings showing the extent of HUMSCs migration after implantation in rat spinal cord with time, wherein the black dots represent the cells of anti-human specific nuclear-antigen positive cells.

FIG. 7 comprises FIGS. 7A-7C and are images showing the non-differentiation of HUMSCs in rat spinal cord.

FIG. 8 is a plot showing the comparison of the open-field locomotion scores between the control and stem cell groups quantified with the BBB score, wherein * represents p<0.05.

### DETAILED DESCRIPTION OF THE INVENTION

Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. In this application, certain terms are used frequently, which shall have the meanings as set in the specification. It must be noted that as used herein and in the appended claims, the singular forms “a,” “an,” and “the” include plural reference unless the context clearly dictates otherwise.

Embodiments of the present invention comprise methods of treating a spinal injury in a subject, comprising delivering to an area of the spinal injury an effective amount of HUMSCs. As used herein, the term “human umbilical mesenchymal stem cells” or “HUMSCs” refers to stem cells originated from the mesenchymal tissue in the human umbilical cord, which includes the Wharton's Jelly, a gelatinous substance within the umbilical cord. The term “HUMSCs” encompasses not only stem cells isolated directly from the mesenchymal tissue in human umbilical cord, but also stem cells cultivated in vitro from the stem cells isolated directly from the mesenchymal tissue in human umbilical cord. HUMSCs are multipotent and can differentiate into a variety of cell types, such as osteoblasts, chondrocytes, myocytes, adipocytes, beta-pancreatic islets cells and neuron-like cells.

HUMSCs can be used as donor cells for transplantation into the nerve system, because they are (i) easily available; (ii) capable of rapid expansion in culture; (iii) immunologically compatible; (iv) capable of long term survival and integration in the host nerve system, and (v) amenable to stable transfection and long-term expression of exogenous genes, which are required characteristics for ideal donor cells for clinical transplantation (Bjorklund et al., Nature 1993; 362 (6419): 414-5).

As used herein, the term “spinal injury”, also referred to as “spinal cord injury”, refers to a damage or myelopathy to the spinal cord of a subject, including damage to white matter or myelinated fiber tracts that carry sensation and motor signals to and from the brain and damage to gray matter in the central part of the spinal cord, causing segmental losses of interneurons and motorneurons. The spinal injury can be a complete injury or an incomplete injury. In a complete injury, there is no motor or sensory function preserved in the sacral segments S4-S5, the lowest spinal cord level representing the anal sphincter and peri-anal sensation. In an incomplete injury, some sensation or movement below the level of the injury is preserved.

The spinal injury can be caused by many reasons, including, but not limited to, trauma, such as vehicle accidents, falls, gunshots, war injuries; tumors, such as meningiomas, ependymomas, astrocytomas, and metastatic cancer; ischemia resulting from occlusion of spinal blood vessels, including dissecting aortic aneurisms, emboli, arteriosclerosis; developmental disorders, such as spina bifida, meningomyolcoele, and other; neurodegenerative diseases, such as Friedreich's ataxia, spinocerebellar ataxia, etc.; demyelinative diseases, such as multiple sclerosis; transverse myelitis, resulting from spinal cord stroke, inflammation, or other causes; vascular malformations, such as arteriovenous malformation (AVM), dural arteriovenous fistula (AVF), spinal hemangioma, cavernous angioma and aneurysm.

In a preferred embodiment of the present invention, the spinal injury is a complete spinal cord transection.

As used herein, the term “subject” refers to an animal, preferably a mammal, most preferably a human, who has been the object of treatment, observation or experiment. Examples of a subject can be a human, a livestock animal (beef and dairy cattle, sheep, poultry, etc.), a companion animal (dog, cat, horse, etc.), or a race animal, e.g., a race horse.

As used herein the term “treatment”, “treat” or “therapy” refers to the prevention of deterioration of a disease, disorder or condition when a patient contracts such a disease, disorder or condition, preferably, at least maintenance of the status quo, and more preferably, alleviation, still more preferably, resolution of the disease, disorder or condition. As used herein, the term “treat” in the broadest sense, with respect to a disease, disorder or condition, refers to any medical act relating thereto, and includes any act for diagnosis, therapy, prevention, prognosis and the like. As used herein, “treating a spinal injury” also includes aiding recovery from spinal injuries.

When used for treating a spinal injury, HUMSCs can be used before, simultaneously with or after the spinal injury. Those skilled in the art will be able to use an effective amount of HUMSCs for the treatment of a spinal injury.

The term “effective amount” as used herein, means that amount of HUMSCs that elicits the biological or medicinal response in a tissue system of a subject, or in a subject, that is being sought by a researcher, veterinarian, medical doctor or other clinician, which includes interdicting, preventing, palliating, or alleviating a syndrome or a condition of discomfort of the subject associated with the spinal injury being treated. The administration of an effective amount of HUMSCs to an area of the spinal injury in the subject results in a clinically observable beneficial effect. The clinically observable beneficial effect can be a situation that an observable syndrome or condition of discomfort associated with the spinal injury is prevented from further development or aggravation or develop to a lesser degree, than without administration of the composition of the present invention.

The syndrome or condition of discomfort of the subject associated with the spinal injury being treated varies according to the type and level of the injury. In addition to a loss of sensation and motor function below the point of injury, individuals with spinal cord injuries will often experience other complications of spinal cord injury, such as dysfunction of the bowel and bladder, including infections of the bladder, and anal incontinence; sexual dysfunction; loss of breathing, necessitating mechanical ventilators or phrenic nerve pacing; inability or reduced ability to regulate heart rate and/or blood pressure, sweating and hence body temperature; spasticity (increased reflexes and stiffness of the limbs); neuropathic pain; autonomic dysreflexia or abnormal increases in blood pressure, sweating, and other autonomic responses to pain or sensory disturbances; atrophy of muscle; osteoporosis (loss of calcium) and bone degeneration; and gall bladder and renal stones.

In one embodiment, the observable beneficial effect is a complete recovery from the spinal injury. In another embodiment, the observable beneficial effect is an improvement in the pathological conditions of the subject suffering from a spinal injury. In yet another embodiment, the observable beneficial effect is a restoration, completely or partially, of the physical function of the injured spinal cord.

Methods are known in the art for determining therapeutically effective doses of HUMSCs according to the present disclosure. A useful assay for confirming an effective amount (e.g., a therapeutically effective amount) for a predetermined application is to measure the degree of recovery from a target disease, disorder or condition. An amount of HUMSCs actually transplanted to the area of spinal injury varies in view of many parameters, such as the condition of the subject, the type and severity of the spinal injury, the route of transplantation, e.g., direct or indirect, etc. The amount of HUMSCs, when applied to the subject suffering from spinal injury, should attain a desired effect, i.e., repairs spinal injury and/or enhances at least partially functional recovery of the injured spinal cord, without significant side effects.

The determination of a therapeutically effective amount is within the ability of those skilled in the art in view of the present disclosure. In one embodiment, a therapeutically effective amount can be estimated using any appropriate animal model. The animal model is used to achieve a desired concentration range and an administration route. Thereafter, such information can be used to determine a dose and route useful for administration into humans. For example, in the examples of the present invention, the spinal cords of rats were completely transected, an effective amount of HUMSCs resulted in recovery from the spinal injury, i.e., regeneration of the severed spinal cord and reduction in motor deficits. Information from such animal studies would guide clinical studies in humans.

The exact dose of HUMSCs is chosen by an individual physician in view of the condition of a patient to be treated. Doses and administration are adjusted to provide a sufficient level of the active portion, or to attain a desired effect. Preferably, a therapeutically effective amount of HUMSCs will reduce a syndrome or a condition of discomfort of the subject associated with the spinal injury under treatment by at least about 20%, for example, by at least about 30%, about 40%, about 50%, about 60%, about 70%, about 80%, about 90%, or about 100%.

In a preferred embodiment of the present invention, the effective amount of HUMSCs delivered to the area of spinal injury is about 10⁴ to about 10⁷ cells per administration.

To transplant HUMSCs to the area of spinal injury, the HUMSCs can be directly delivered to an exposed area of the spinal injury by means of injection. The HUMSCs can also be delivered to the area of the spinal injury by means of a suitable vehicle. In a preferred embodiment of the present invention, the HUMSCs are delivered to the desired area together with a fibrin glue.

The term “fibrin glue” as used herein refers to a biocompatible and biodegradable product comprising fibrinogen and other reagents. Fibrin glue has been used as an adhesive agent in various kinds of surgery, including neurosurgery. It is commercially available, from example, under the trademark Beriplast P™ (ZLB Behring, Germany). In a preferred embodiment, the fibrin glue used in embodiments of the present invention comprises fibrinogen, aprotinin and a calcium source that provides divalent calcium ions (such as calcium chloride or calcium carbonate).

Where fibrin glue is used, HUMSCs can be delivered, before, simultaneously or after the delivery of a fibrin glue to an area of the spinal injury. In one embodiment, HUMSCs can be mixed with fibrinogen and aprotinin before mixing with calcium chloride in the surgical area. For example, fibrinogen is first mixed with aprotinin, and further mixed with the calcium source in the surgical area to form a glue cast.

In a fibrin glue used in the present invention, the concentration of fibrinogen can preferably be in the range of about 10 mg/ml to about 1000 mg/ml, and more preferably about 100 mg/ml; the concentration of aprotinin can preferably be in the range of about 10 KIU/ml to about 500 KIU/ml, more preferably about 200 KIU/ml; and the concentration of calcium chloride used as the calcium source can preferably be in the range of about 1 mM to about 100 mM, more preferably about 8 mM.

In another embodiment of the present invention, transplantation of HUMSCs to the area of spinal injury is achieved by using a hollow conduit filled with HUMSCs to bridge the gap between severed ends of a transected spinal cord.

The conduit used in the present invention is preferably composed of a biodegradable polymeric material conventionally used to make nerve conduits, including but not limited to, collagen, poly(lactic acid) (PLA), poly(glycolic acid) (PGA), poly(lactic-co-glycolic acid) (PLGA), polycaprolactone, poly(caprolactone-co-lactic acid) (PCLA), chitosan, alginate, hyaluronic acid, gelatin, and fibrin.

The conduit used in the present invention can be any commercially available conduit composed of a biodegradable polymeric material. Alternatively, the hollow conduit can be fabricated as needed by any known method, such as the fiber templating process of Flynn et al. as set forth in Biomaterials 24: 4265-4272 (2003), and the low-pressure injection molding process of Sundback et al. as set forth in Biomaterials 24: 819-830 (2003); contents of both are incorporated herein by reference as if set out in full.

In use, the severed ends of a transected spinal cord are brought into contact with the respective ends of a hollow conduit filled with HUMSCs, which is slightly longer than the gap to be bridged, so that no tension is placed upon the severed spinal cord. Both the distal and proximal ends of the spinal cord are partially inserted into the conduit. If necessary, the severed ends can be sutured to the conduit over their perineurium.

In another embodiment of the present invention, an effective amount of HUMSCs are delivered to an area of a spinal injury in combination with one or more additional treatments for the spinal injury. For example, HUMSCs can be used together with an effective dose of methylprednisolone, a treatment for acute traumatic spinal cord injuries. HUMSCs can also be used together with chondroitinase treatment and other stem cell transplants. HUMSCs can further be used in combination with Schwann cells bridge (SCs bridge), olfactory ensheathing glia and chondroitinase ABC. The effective amount of HUMSCs can be combined with one or more additional treatments for the spinal injury in view of the present disclosure and the methods known to those skilled in the art.

The present invention is further illustrated by the following examples, which are provided for the purpose of demonstration rather than limitation.

### EXAMPLES

### Preparation of HUMSCs from Wharton's Jelly

Human umbilical cords were collected in HBSS (Gibco 14185-052, USA) at 4° C. Following disinfection in 75% ethanol for 30 seconds, the umbilical cord vessels were cleared off while still in HBSS. The mesenchymal tissue in Wharton's jelly was then diced into cubes of about 0.5 cm³ and centrifuged at 250 g for 5 min. Following removal of the supernatant fraction, the precipitate (mesenchymal tissue) was washed with serum-free DMEM (Gibco 12100-046) and centrifuged at 250 g for 5 min. Following aspiration of the supernatant fraction, the precipitate (mesenchymal tissue) was treated with collagenase at 37° C. for 18 h, washed, and further digested with 2.5% trypsin (Gibco 15090-046) at 37° C. for 30 minutes. Fetal bovine serum (FBS; Hyclone SH30071.03, USA) was then added to the mesenchymal tissue to neutralize the excess trypsin. The dissociated mesenchymal cells were further dispersed by treatment with 10% FBS-DMEM and counted under the microscope with the aid of a hemocytometer. The mesenchymal cells were then used directly for cultures or stored in liquid nitrogen for later use.

Experimental Animals

For the different treatment, the rats were divided into two experimental groups: (1) control group with transection bridged with the fibrin glue-only bridge (n=8); and (2) stem cell group with transection bridged with the HUMSCs fibrin-glue bridge and rostrocaudal HUMSC grafts (n=13).

Spinal Cord Transection and HUMSCs Grafting

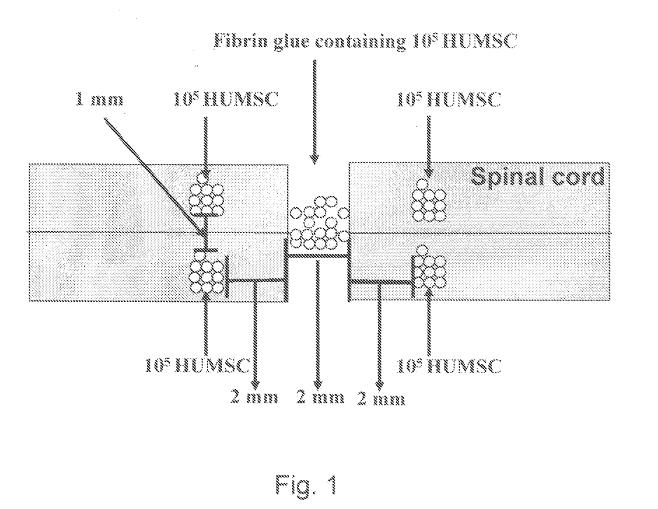

After a laminectomy at the T7-T9 vertebral level, the meningeal membranes were severed along with the dura mater, the spinal cord was completely transected, and a 2 mm region of spinal cord encompassing T8 was removed. The rostral and caudal stumps were lifted after removal of the spinal cord segment to ensure complete discontinuity. Thereafter, 10⁵ HUMSCs were drawn into a glass pipette with a tip diameter of 150-200 μm mounted onto a 5 μl Hamilton syringe (Hamilton, Reno, Nev.) attached to a micromanipulator. The cells were deposited into two injection sites at the rostral as well as the caudal stump, 2 mm from the lesion, at 500 μm lateral to the midline, and to a depth of 1000 μm. A volume of 5 μl containing 10⁵ HUMSCs in PBS was grafted into each site. Next, 10⁵ HUMSCs in a fibrin glue carrier were implanted to bridge the 2-mm gap to fill the channel in the severed spinal cords. The experimental design is illustrated in FIG. 1.

Determination of Basso, Beattie, and Bresnahan (BBB) Score

The open-field locomotor score (Basso et al., A sensitive and reliable locomotor rating scale for open field testing in rats. J Neurotrauma 1995; 12: 1-21) was determined by two blinded observers. Before testing, bladders were expressed, because spontaneous bladder contraction often accompanies hindlimb activity. The rats were placed in an open field and were observed for 3 min. Hindlimb movements immediately after contact with experimenters were disregarded. During the open-field activity, the animals were also video-monitored.

Histological Study of Spinal Cord

Fixation and Sectioning

The rats were perfused with fixation solution (4% paraformaldehyde and 7.5% piric acid in 0.1M phosphate buffer (PB) 4 months after surgery. Spinal cord was taken out and immersed in fixation solution at 4° C. for 24 hours and then switched to phosphate buffered saline (PBS) containing 30% sucrose before cryosection. Successive sections were sliced by cryo-microtome from spinal cord tissues at a thickness of 30 μm and adhered onto gelatin-pretreated slides.

NF, GFAP and MBP Immunostaining

Immunohistochemistry was performed by using primary antibody against neurofilament 60 kD (NF, Chemicon, 1:500), glial fibrillary acidic protein (GFAP, Chemicon, 1:1000) and ED1 (Chemicon, 1:500) as well as secondary antibodies (Biotin-conjugated goat anti-rabbit-IgG, 1:300 diluted, Sigma and Biotin-conjugated goat anti-mouse-IgG, 1:300 diluted, Sigma), followed by avidin-biotinylated-horseradish peroxidase complex (ABC KIT, Vectorlabs PK-4000) and 3,3′-diaminobenzidine (5 mg DAB, 3.5 μl 30% H₂O₂ in 10 ml 50 mM Tris Buffer). Tissue sections were dehydrated and mounted with Permount™ mounting media. Pathological changes and variations in the nerve fiber count in the bridge of the spinal cord were observed under optical microscope and analyzed by the Image-Pro® software from Cybernetics, Inc.

Double Staining of Anti-Human-Specific Nuclear Antigen and Anti-NF or Anti-GFAP or Anti-MBP

For the assessment of the possible differentiation of HUMSCs into subpopulations of neurons, astrocyte or oligodendrocyte, double staining for human-specific nuclear antigen and neurofilament, GFAP, and MBP were performed.

Spinal cord sections were fixed with 4% paraformaldehyde in 0.1 M phosphate buffer for 20 min and then washed with 0.1 M phosphate buffer. They were then treated with a blocking solution for 30 min in order to prevent nonspecific antibody-antigen binding. The sections were then reacted with primary antibodies against neurofilament 60 kD (NF, Chemicon, 1:500), glial fibrillary acidic protein (GFAP, Chemicon, 1:1000) and MBP (Chemicon, 1:500) at 4° C. for 18 h, washed with 0.1 M PBS, and then reacted with secondary antibodies (alkaline phosphatase-conjugated goat anti-mouse-IgG for human nuclei, 1:50; Biotin-conjugated-goat-anti-mouse-IgG, 1:200, or Biotin-conjugated-goat-anti-rabbit-IgG, 1:200) at room temperature for 1 h. The sections were then observed under a microscope. In order to trace the distribution of HUMSCs, the stained tissues were visualized under a microscope for mapping of HUMSCs.

Corticospinal Tract Staining

At 16 weeks after injury, the corticospinal tract was bilaterally traced under anesthesia. For the corticospinal tract tracing, 3 μl of the tracer biotinylated dextran amine (BDA, MW=10,000, D-1956, 10% in 0.01 M PBS, Molecular Probes, Eugene, Oreg.) was used. Two weeks after the tracing, the animals were killed with pentobarbital and transcardially perfused with phosphate-buffered 4% paraformaldehyde (0.1 M, pH 7.4). The spinal cord and brain were removed and postfixed overnight in the same fixative at 4° C. The spinal cord was cryoprotected in 30% sucrose in PB (0.1 M, pH 7.4) for at least 24 h, frozen in Tissue-Tek OCT compound (Sakura Finetek, Torrance, Calif.), and cut into 30 μm-thick sagittal sections. Sections were then processed with avidin-HRP (ABC Elite; Vector Laboratorie) followed by a diaminobenzidine HRP reaction for the visualization of the BDA tracer.

Statistical Analyses

All data were presented as mean±standard error (SE). Two-way ANOVA was used to compare all means and Least Significant Difference (LSD) was used for the posteriori test. In all statistical analyses, p<0.05 was considered significant.

Promotion of Reconnection of Corticospinal Fiber to Cross the Bridge after HUMSCs Transplantation

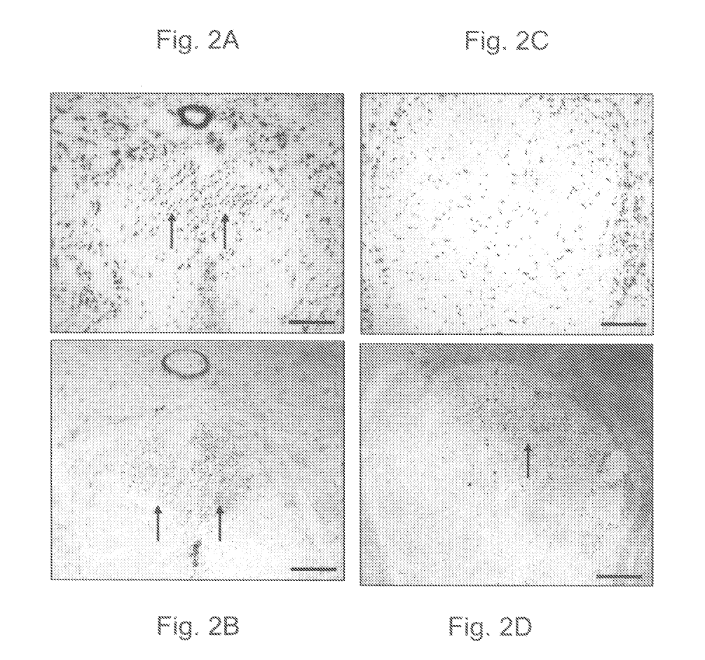

In order to trace whether the cortico-spinal fibers has passed the bridge of the transection, tracer BDA was injected into the motor cortices of the rats 16 weeks after the surgery. The results are shown in FIGS. 2A-2D, which are the enlarged images of the cross-sections of the dorsal column. FIGS. 2A and 2B show the rostral stumps of the spinal cord. FIGS. 2C and 2D show the caudal stumps.

In the control groups, BDA positive labeled dots appeared on the rostral stumps of the transections (FIG. 2A), but not on the caudal stumps (FIG. 2C). This demonstrates that in the control group, the cortico-spinal fibers had not grown through the transected bridge. In the stem cell groups, both rostral and caudal stumps of the spinal cord had patches of BDA positive fibers (FIGS. 2B and 2D, indicated by arrows). These BDA positive fibers also extended up to 1 cm caudal to the bridge. However, the number of nerve fibers which had extended through the bridge to the caudal stumps was significantly lower than the number at the rostral stumps. This indicates that in the stem cell group the corticospinal fibers grew through the transection and extended into the lumbar segments of the spinal cord.

Increase in Regeneration of Neuronal Axons in the Bridge after HUMSCs Transplantation

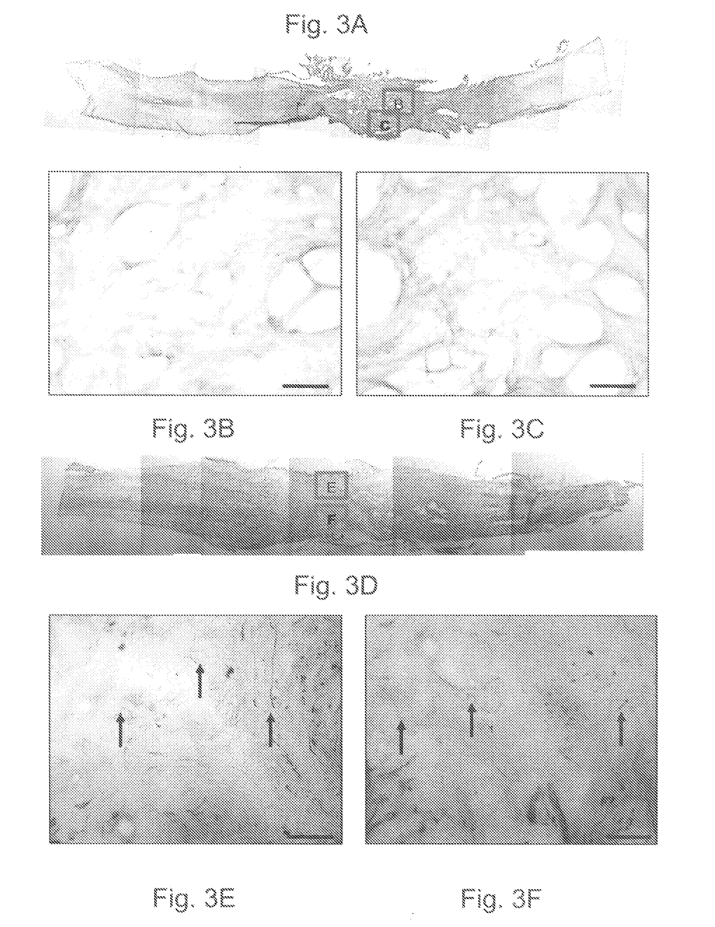

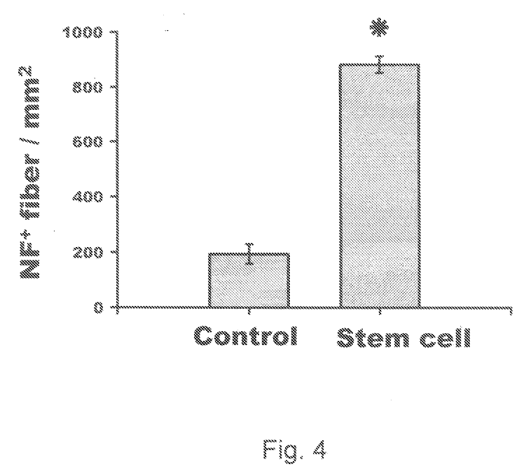

Anti-neurofilament immunostaining was used to label the neurofilament. A quantitative analysis of the number (A) and length (B) of the neurofilament positive fibers was carried out. The quantification of neuronal axons was performed in spinal sections obtained from the center of the bridge. These counts likely include both descending and ascending fibers that cannot be distinguished by this approach. The lowest count and total length of NF positive axons were found in the control group with an average of (193.6±35.3) counted axons/mm² and (2839.1±802.1) μm/mm² (FIGS. 3A-3C and FIG. 4). In the stem cell group, the average numbers were 882.3±30.1 counted axons/mm² and 10184.4±669.8 μm/mm² which were statistically increased compared to that of control group (FIGS. 3D-3F and FIG. 4).

In summary, the neurofilament-positive fibers were found to grow in the channel of transection spinal cord in the stem cell group (FIGS. 3D-3F); and the neurofilament-positive fiber was rarely found in the channel of transection spinal cord in the control group (FIGS. 3A-3C). The number (FIG. 4) and length of nerve fibers were significantly higher in the stem cell group than in the control group (* represents p<0.05).

Survival and Migration Pattern of HUMSCs in Implanted Spinal Cord

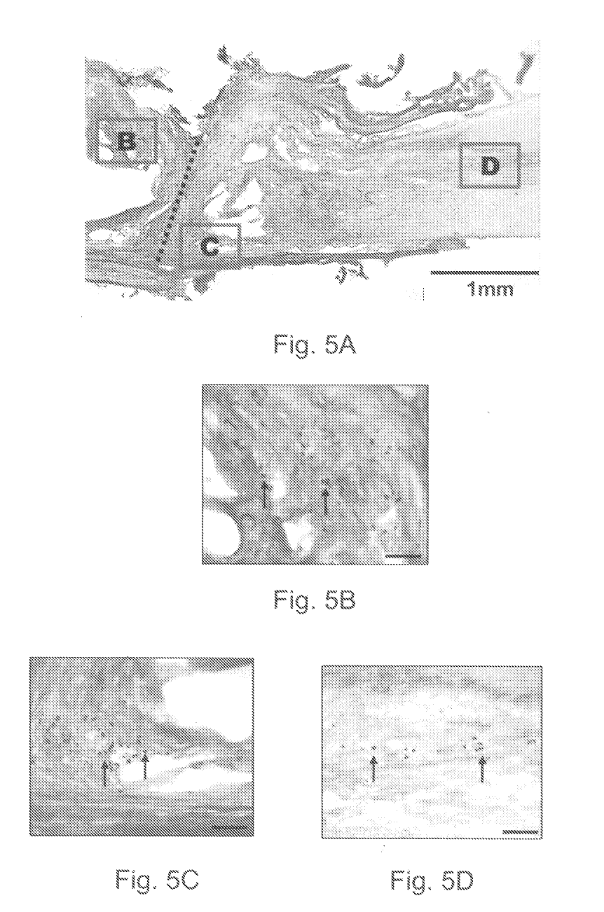

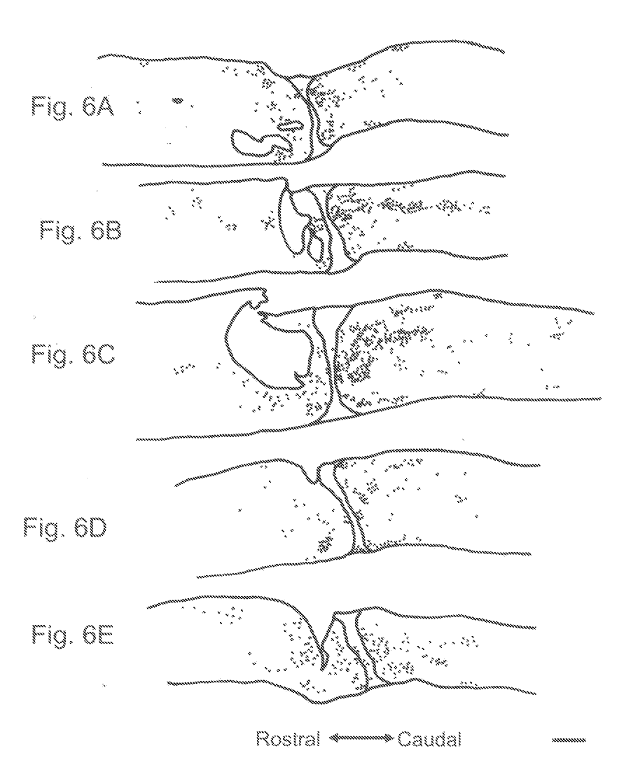

Anti-human specific nuclear antigen immunostaining was used to trace the survival rate and migration pattern of HUMSCs. In the stem cell group, HUMSCs survived around the implantation sites of the rostral stump, bridge, and caudal stump (FIGS. 5A-5D). The series of sections showed that a large numbers of HUMSCs survived at least for 4 months after transplantation. The HUMSCs had migrated for about 1.5 mm in caudal direction of the rostrocaudal axis from the implantation site (FIGS. 6A-6E).

Non-Differentiation of Transplanted HUMSCs in Rat Spinal Cord

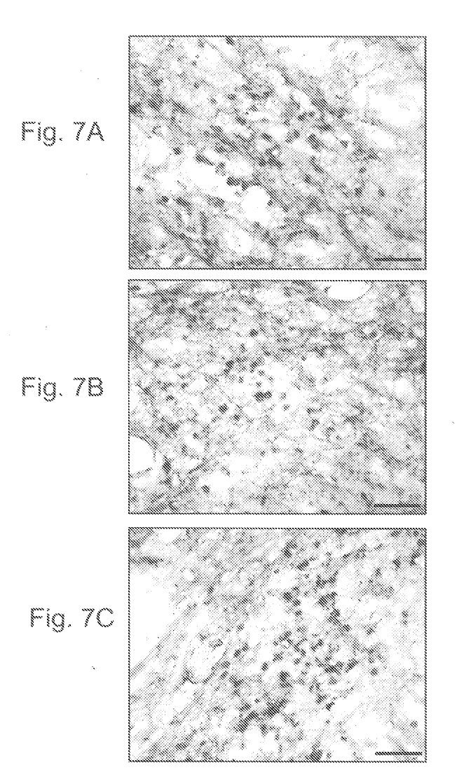

Double-staining of human-specific nuclear antigen and NF revealed that most HUMSCs did not differentiate into NF-positive fibers (FIG. 7A). Staining with anti-human specific nuclear antigen and anti-GFAP showed that majority of HUMSCs had not differentiated into astrocytes (FIG. 7B). Double-staining of human-specific nuclear antigen and MBP, indicating that most HUMSCs were MBP negative cells, suggesting HUMSCs did not differentiate into oligodendricyte (FIG. 7C).

Improvement in Locomotor Recovery after HUMSCs Transplantation

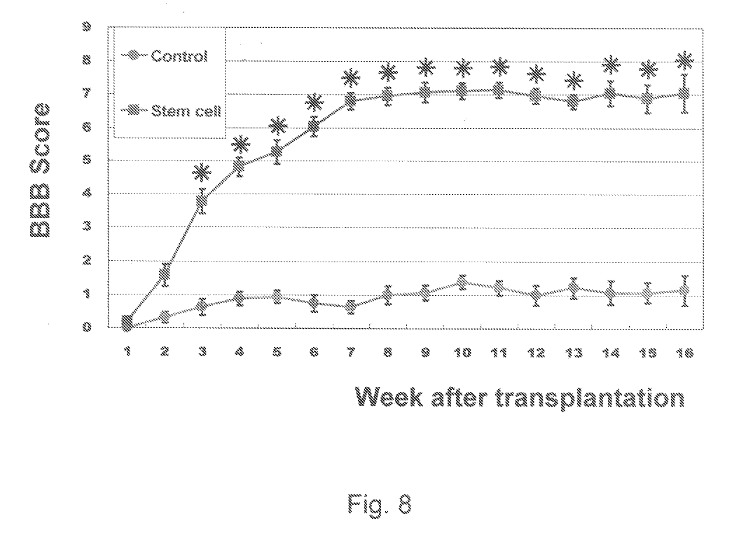

Behavioral testing was performed until 16 weeks post-lesion. In the control groups, no significant difference was seen until 16 weeks after injury. Their BBB scores ranged between 0 and 2 points. The first signs of recovery in locomotor functions were observed and were statistically significant between the stem cell group and the control group, at 3 weeks. Among the stem cell groups, BBB scores rose to 6-8 points by week seven. This trend continued up to 16 weeks (FIG. 8, p<0.05 two way ANOVA followed by LSD test). Rats in the stem cell groups were able to coordinate movement between the forelimbs and the three joints of the hindlimbs to achieve a walk. They were also able to lift their bodies off the ground for brief periods, although they were unable to raise their body weight while walking.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method for treating a spinal injury in a subject comprising delivering an effective amount of human umbilical mesenchymal stem cells (HUMSCs) to an area of the spinal injury.

2. The method of claim 1, wherein the HUMSCs are obtained from Wharton's Jelly.

3. The method according to claim 1, wherein the HUMSCs are delivered by direct injection into the area of the spinal injury.

4. The method according to claim 1, wherein the HUMSCs are delivered to the area of the spinal injury together with a fibrin glue.

5. The method according to claim 4, wherein the fibrin glue comprises about 10 mg/ml to about 1000 mg/ml fibrinogen, about 10 KIU/ml to about 500 KIU/ml aprotinin; and about 1 mM to about 100 mM calcium.

6. The method according to claim 5, wherein the fibrin glue comprises about 100 mg/m fibrinogen, about 200 KIU/ml aprotinin and about 8 mM calcium chloride.

7. The method according to claim 4, wherein the HUMSCs are delivered before, simultaneously or after the delivery of the fibrin glue to the area of the spinal injury.

8. The method according to claim 1, wherein the HUMSCs are delivered in a hollow conduit filled with the HUMSCs.

9. The method according to claim 8, wherein the hollow conduit bridges a gap between severed ends of a transected spinal cord.

10. The method according to claim 8, wherein the hollow conduit comprises a biodegradable polymeric material selected from the group consisting of collagen, poly(lactic acid) (PLA), poly(glycolic acid) (PGA), poly(lactic-co-glycolic acid) (PLGA), polycaprolactone, poly(caprolactone-co-lactic acid) (PCLA), chitosan, alginate, hyaluronic acid, gelatin, and fibrin.

11. The method according to claim 1, wherein the HUMSCs are delivered in combination with at least one additional treatment.

12. The method according to claim 11, wherein the at least one additional treatment is selected from the group consisting of methylprednisolone treatment, chondroitinase treatment, transplantation of another stem cell, Schwann cells bridge (SCs bridge), olfactory ensheathing glia and a combination thereof.

13. The method according to claim 1, wherein the effective amount of HUMSCs is about 10⁴ to about 10⁷ cells per administration.

14. The method according to claim 1, wherein the spinal injury is a complete injury.

15. The method according to claim 14, wherein the complete injury is a transection of the spinal cord.

16. The method according to claim 1, wherein the spinal injury is an incomplete injury.

17. The method according to claim 1, wherein the HUMSCs are delivered before, simultaneously or after the spinal injury.
